SEQUENCE LISTING (4 sequences)

SEQ ID NO: 1 - protein, 161 aa, synthetic construct
MVPTPQSDLPLNENDSQEMVLYEVLNEANALNIPYLPQRNQLLPRNNILRPLQCIGKKYRGVRRRPWGKYAAEIRDSARHGARIWLGTFETAEEAALAYDRAAFRMRGAKALLNFPSEIVNASVSVDKLSLCSNSYTTNNNSDSSLNEVSSGTNDVFESRC

SEQ ID NO: 2 - protein, 86 aa, Capsicum annuum
MVSKSSIFICLSLIILLIMSTQIVAREMTSEASASLTQAMNGNNISETKKVGRHLVKGLGKIFKAGKVIYCKTCKTCHGRCDYCCA

SEQ ID NO: 3 - DNA, 486 bp, synthetic construct
  source 1..486: mol_type = other DNA
atggttccaactccacagtctgatcttccactcaacgagaacgattctcaagagatggttctttacgaggttttgaacgaggctaacgctcttaacattccatacctcccacaaagaaaccagcttctccctaggaacaacattcttaggccacttcagtgcattggaaagaagtacaggggagttagaagaaggccttggggaaagtacgctgctgagattagagattctgctagacacggtgctagaatttggcttggaactttcgaaactgctgaagaagctgctcttgcttacgatagagctgctttcagaatgagaggtgctaaggctcttttgaacttcccatccgagattgtgaacgcttctgtgtctgtggataagctttctctttgctccaactcttacaccaccaacaacaactctgattctagccttaacgaggtttcctctggaactaacgatgttttcgagtccaggtgctaa

SEQ ID NO: 4 - DNA, 261 bp, Capsicum annuum
  source 1..261: mol_type = genomic DNA
atggttagtaagtctagtatctttatctgccttagcctgattatcctcctgattatgagcactcagatcgtggctagggaaatgactagtgaggctagcgctagtctgactcaggctatgaacggtaacaatattagcgagactaagaaggtgggccgtcaccttgttaagggactcggtaagatctttaaggccggtaaggtgatctactgtaagacttgtaagacctgtcacggtaggtgcgattactgctgcgcttaa

(19) United States
(12) Patent Application Publication
(10) Pub. No.: US 2026/0201404 A1
(43) Pub. Date: Jul. 16, 2026

(54) BROAD FUNGAL PATHOGEN RESISTANCE IN CROP PLANTS

(71) Applicant: BASF AGRICULTURAL SOLUTIONS US LLC, Research Triangle Park, NC (US)

(72) Inventors: Yunxing Cory Cui, Durham, NC (US); Renata Bocci Zanon, Santo Antonio De Posse (BR); Marianela Rodriguez, Durham, NC (US); Jingkai JK Zhou, Nevada, IA (US); Brody John De Young, Chapel Hill, NC (US); Holger Schultheiss, Ludwigshafen (DE)

(21) Appl. No.: 19/138,264

(22) PCT Filed: Dec. 8, 2023
(86) PCT No.: PCT/EP2023/084959

§ 371 Date: Jun. 12, 2025

Related U.S. Application Data
(60) Provisional application No. 63/387,469, filed on Dec. 14, 2022.

(51) Int. Cl. C12N 15/82; A01H 1/04; C07K 14/415; C12Q 1/6895

(52) U.S. Cl. CPC C12N 15/8282; A01H 1/045; C07K 14/415; C12Q 1/6895; C07K 2319/00; C12Q 2600/13

(57) ABSTRACT

The present invention relates to plant breeding and farming. In particular, the invention relates to materials and methods for improving plant resistance to multiple plant pathogens.

### FIELD OF THE INVENTION

The present invention relates to plant breeding and farming. In particular, the invention relates to materials and methods for improving plant resistance to multiple plant pathogens.

### BACKGROUND OF THE INVENTION

Plant pathogenic organisms, in particular fungi, have resulted in severe reductions in crop yield in the past, in worst cases leading to famine. Monocultures, in particular, are highly susceptible to an epidemic-like spreading of diseases. To date, the pathogenic organisms have been controlled mainly by using pesticides. Currently, the possibility of directly modifying the genetic disposition of a plant or pathogen is also open to man. Alternatively, naturally occurring fungicides produced by the plants after fungal infection can be synthesized and applied to the plants.

Fungi are distributed worldwide. Approximately 100 000 different fungal species are known to date. Thereof, rusts are of great importance. They can have a complicated development cycle with up to five different spore stages (spermatium, aecidiospore, uredospore, teleutospore and basidiospore). Specific infection structures are developed for penetration of the plant. Biotrophic phytopathogenic fungi depend on the metabolism of living plant cells for their nutrition. Examples of biotrophic fungi include many rust fungi, powdery mildew fungi or oomycete pathogens like the genus Phytophthora or Peronospora. Necrotrophic phytopathogenic fungi depend for their nutrition on dead cells of the plants, e.g. species from the genus Fusarium, Rhizoctonia or Mycosphaerella. Soybean rust occupies an intermediate position. It penetrates the epidermis directly, whereupon the penetrated cell becomes necrotic. However, after penetration, the fungus changes over to an obligate-biotrophic lifestyle. The subgroup of the biotrophic fungal pathogens which follows essentially such an infection strategy are heminecrotrophic.

In the past efforts have been made to create plants resistant against biotic stresses such as fungal pathogens. The term “resistance” as used herein refers to an absence or reduction of one or more disease symptoms in a plant caused by a plant pathogen. Resistance generally describes the ability of a plant to prevent or at least curtail the infestation and colonization by a harmful pathogen. Different mechanisms can be discerned in the naturally occurring resistance, with which the plants fend off colonization by phytopathogenic organisms (Schopfer and Brennicke (1999) Pflanzenphysiologie, Springer Verlag, Berlin-Heidelberg, Germany). In nature, however, resistance is often overcome because of the rapid evolutionary development of new virulent races of the pathogens, including fungi (Neu et al. (2003) American Cytopathol. Society, MPMI 16 No. 7:626-633).

A multitude of genes/proteins increasing plant resistance to a specific pathogen has been published. Often those publications could only show that the plant becomes more resistant against a single pathogen under artificial conditions (e.g. greenhouse, phytochamber), neglecting the complex interaction of plants growing in the field with multiple pathogens and other microbes.

For example, in most years Asian soybean rust (Phakopsora pachyrhizi) is the most prominent disease in southern Brazil, whereas target spot (caused by Corynespora cassiicola), is becoming a bigger problem in the north-eastern parts of Brazil. In addition, several so called “late cycle” or “final cycle” diseases, such as Septoria brown spot (Septoria glycines) or Cercospora Leaf Blight (Cercospora kukuchii and other related species) are threatening the soybean crop late in the season, when the maturing pods are filled. In contrast to Brazil, soybean in US is mainly endangered by diseases like Frogeye leaf spot disease (Cercospora sojina), Sudden death syndrome (SDS, Fusarium virguliforme) and Phyllosticta leaf spot (caused by the fungus Phyllosticta sojicola). A more complete overview of soybean diseases and the respective symptoms and disease management can be found in many publications, e.g. Glen L. Hartman et al. (2015), Compendium of Soybean Diseases and Pests.

On the molecular level disease resistance of plants is a complex and strictly regulated network. The most prominent defense inducing and regulating pathways are the salicylic acid (SA) and jasmonic acid (JA)/ethylene (ET)-mediated signalling pathways, which together represent the backbone of plant defense against diseases.

In general, salicylic acid mediated defense pathways are involved in resistance to biotrophic pathogens, such as powdery mildew and many rust fungi, and ethylene/jasmonic acid mediated defense is contributing to the defense against necrotrophic pathogens. To balance resistance against the different classes of pathogens, it has been shown that salicylic acid and ethylene/Jasmonic acid defense pathways are antagonistic and inhibit each other (for review see: Li N, Han X, Feng D, Yuan D, Huang L J. Signaling Crosstalk between Salicylic Acid and Ethylene/Jasmonate in Plant Defense: Do We Understand What They Are Whispering? Int J Mol Sci. 2019 Feb. 4; 20(3):671).

Therefore, increased resistance to biotrophic pathogens is mostly linked to an increased susceptibility to necrotrophic pathogens and vice versa. Therefore, broad resistance to multiple diseases with different lifestyles is difficult to achieve.

There is thus a need to provide material and method to prevent emergence or reduce the severity of multiple diseases with different lifestyles simultaneously in a crop plant. In particular, there is a need to prevent emergence or reduce the severity of diseases caused by all of biotrophic, heminecrotrophic and necrotrophic pathogens in soybean.

### SUMMARY OF THE INVENTION

The invention thus provides a method for conferring, modifying or increasing resistance against one or multiple pathogen infections of a plant, plant part, or plant cell in comparison to a respective wild type plant, wild type plant part, or wild type plant cell, wherein the pathogens are selected from
the following taxa: (1) order Pleosporales, preferably family Corynesporascaceae, (2) order Mycosphaerellales, preferably family Mycosphaerellaceae, (3) order Erysiphales, preferably family Erysiphaceae, (4) order Botryosphaeriales, preferably family Phyllostictaceae and/or
the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot,
   wherein the method comprises the step of increasing the production and/or accumulation of a combination of at least a Pti5 protein and a CaSAR protein, and/or a Pti5-CaSAR fusion protein, in the plant, plant part or plant cell.

The invention also provides a method of producing a crop plant showing improved resistance against multiple pathogens as described herein compared to the corresponding wild type, comprising the steps of
i) crossing two different crop plants, wherein at least one of the plants comprises a Pti5 gene and a CaSAR gene, and/or a Pti5-CaSAR fusion gene, to produce members of a filial generation,
ii) detecting the presence of a polynucleotide indicative of the Pti5, CaSAR and/or Pti5-CaSAR fusion protein and in said members of the filial generation, and
iii) selecting at least one member of the filial generation with confirmed presence of the polynucleotide indicative of the Pti5, CaSAR and/or Pti5-CaSAR fusion protein.

Furthermore, the invention provides a method of reducing or preventing single or multiple pathogen pressure of a field, comprising the step of cultivating, on said field, plants comprising
a) a heterologous expression cassette comprising a Pti5 gene and a heterologous expression cassette comprising a CaSAR gene, and/or
b) a heterologous expression cassette comprising a Pti5-CaSAR fusion gene,
wherein the pathogens are selected from
   the following taxa: (1) order Pleosporales, preferably family Corynesporascaceae, (2) order Mycosphaerellales, preferably family Mycosphaerellaceae, (3) order Erysiphales, preferably family Erysiphaceae, (4) order Botryosphaeriales, preferably family Phyllostictaceae and/or
   the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot.

And the invention provides a use of (i) a combination of a Pti5 gene and a CaSAR gene, (ii) a Pti5-CaSAR fusion protein or (iii) a combination of a Pti5 or CaSAR gene and a Pti5-CaSAR fusion protein for conferring, modifying or increasing resistance against single or multiple pathogen infections of a plant, plant part, or plant cell in comparison to a respective wild type plant, wild type plant part, or wild type plant cell, wherein the pathogens are selected from
the following taxa: (1) order Pleosporales, preferably family Corynesporascaceae, (2) order Mycosphaerellales, preferably family Mycosphaerellaceae, (3) order Erysiphales, preferably family Erysiphaceae, (4) order Botryosphaeriales, preferably family Phyllostictaceae and/or
the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot.

### BRIEF DESCRIPTION OF FIGURES

FIG. 1 shows the maximum target spot (Corynespora cassiicola) disease severity at 4 different locations across Brazil. The natural infestation of the disease was rated at different dates across the season. As the disease onset and progression is highly dependent on the environment, only the data of the date with the maximum disease severity (in wt control) is shown here in light grey. The average rating of 4 independent events expressing the combination of SAR8.2 and Pti5 is shown in dark grey.

It is clearly visible that the expression of the combination of SAR8.2 and Pti5 is significantly reducing the severity of target spot disease in comparison to the wt control, which is grown in parallel.

FIG. 2 shows the maximum Septoria brown spot (Septoria glycines) disease severity at 2 different locations and 2 seasons across Brazil. The natural infestation of the disease was rated at different dates across the season. As the disease onset and progression is highly dependent on the environment, only the data of the date with the maximum disease severity (in wt control) is shown here in light grey. The average rating of 4 independent events expressing the combination of SAR8.2 and Pti5 is shown in dark grey. It is clearly visible that the expression of the combination of SAR8.2 and Pti5 is significantly reducing the severity of Septoria brown spot disease in comparison to the wild type control, which is grown in parallel.

FIG. 3 shows the maximum Cercospora Leaf Blight (Cercospora kukuchii) disease severity at 4 different locations across Brazil. The natural infestation of the disease was rated at different dates across the season. As the disease onset and progression is highly dependent on the environment, only the data of the date with the maximum disease severity (in wt control) is shown here in light grey. The average rating of 4 independent events expressing the combination of SAR8.2 and Pti5 is shown in dark grey. It is clearly visible that the expression of the combination of SAR8.2 and Pti5 is significantly reducing the severity of Cercospora Leaf Blight in comparison to the wild type control, which is grown in parallel.

FIG. 4 shows the maximum powdery mildew (Erysiphe diffusa) disease severity at 2 different locations in Brazil. The natural infestation of the disease was rated at different dates across the season. As the disease onset and progression is highly dependent on the environment, only the data of the date with the maximum disease severity (in wt control) is shown here in light grey. The average rating of 4 independent events expressing the combination of SAR8.2 and Pti5 is shown in dark grey.

It is clearly visible that the expression of the combination of SAR8.2 and Pti5 is significantly reducing the severity of powdery mildew disease in comparison to the wt control, which is grown in parallel.

### Brief Description of Sequences

| SEQ | | |
|---|---|---|
| ID NO. | type | description |
| 1 | aa | Pti5 protein |
| 2 | aa | CaSAR protein |
| 3 | nt | gene coding for the Pti5 protein of SEQ ID NO. 1 |
| 4. | nt | gene coding for the CaSAR protein of SEQ ID NO. 2 |

### DETAILED DESCRIPTION

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind the skilled person understands that the subject matter of the invention is the sum of the individual technical concepts signified herein or expressed, necessarily in a pars-pro-toto way, by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention.

In so far as recourse herein is made to entries in public databases, for example Uniprot and PFAM, the contents of these entries are those as of 2020 May 20. Unless stated to the contrary, where the entry comprises a nucleic acid or amino acid sequence information, such sequence information is incorporated herein.

As used herein, terms in the singular and the singular forms like “a”, “an” and “the” include plural referents unless the content clearly dictates otherwise. Thus, for example, use of the term “a nucleic acid” optionally includes, as a practical matter, many copies of that nucleic acid molecule; similarly, the term “probe” optionally (and typically) encompasses many similar or identical probe molecules. Also as used herein, the word “comprising” or variations such as “comprises” or “comprising” will be understood to imply the inclusion of a stated element, integer or step, or group of elements, integers or steps, but not the exclusion of any other element, integer or step, or group of elements, integers or steps.

As used herein, the term “and/or” refers to and encompasses any and all possible combinations of one or more of the associated listed items, as well as the lack of combinations when interpreted in the alternative (“or”). The term “comprising” also encompasses the term “consisting of”.

The term “about”, when used in reference to a measurable value, for example an amount of mass, dose, time, temperature, sequence identity and the like, refers to a variation of ±0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50% X whilst in other embodiments it may comprise anywhere from 40% to 60% X (i.e., 50%±10%).

As used herein, the term “gene” refers to a biochemical information which, when materialised in a nucleic acid, can be transcribed into a gene product, i.e. a further nucleic acid, preferably an RNA, and preferably also can be translated into a peptide or polypeptide. The term is thus also used to indicate the section of a nucleic acid resembling said information and to the sequence of such nucleic acid (herein also termed “gene sequence”).

Also as used herein, the term “allele” refers to a variation of a gene characterized by one or more specific differences in the gene sequence compared to the wild type gene sequence, regardless of the presence of other sequence differences. Alleles or nucleotide sequence variants of the invention have at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% nucleotide “sequence identity” to the nucleotide sequence of the wild type gene. Correspondingly, where an “allele” refers to the biochemical information for expressing a peptide or polypeptide, the respective nucleic acid sequence of the allele has at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% amino acid “sequence identity” to the respective wild type peptide or polypeptide.

Protein or nucleic acid variants may be defined by their sequence identity when compared to a parent protein or nucleic acid. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

| | |
|---|---|
| Seq A: | AAGATACTG length: 9 bases |
| Seq B: | GATCTGA length: 7 bases |

Hence, the shorter sequence is sequence B.

Producing a pairwise global alignment which is showing both sequences over their complete lengths results in

| | |
|---|---|
| Seq A: | AAGATACTG- |
| | \|\|\| \|\|\| |
| Seq B: | --GAT-CTGA |

The “I” symbol in the alignment indicates identical residues (which means bases for DNA or amino acids for proteins). The number of identical residues is 6.

The “-” symbol in the alignment indicates gaps. The number of gaps introduced by alignment within the sequence B is 1. The number of gaps introduced by alignment at borders of sequence B is 2, and at borders of sequence A is 1.

The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence).

Accordingly, the alignment length showing sequence A over its complete length would be 9 (meaning sequence A is the sequence of the invention), the alignment length showing sequence B over its complete length would be 8 (meaning sequence B is the sequence of the invention).

After aligning the two sequences, in a second step, an identity value shall be determined from the alignment. Therefore, according to the present description the following calculation of percent-identity applies:
%-identity=(identical residues/length of the alignment region which is showing the respective sequence of this invention over its complete length)*100. Thus, sequence identity in relation to comparison of two amino acid sequences according to the invention is calculated by dividing the number of identical residues by the length of the alignment region which is showing the respective sequence of this invention over its complete length. This value is multiplied with 100 to give “%-identity”. According to the example provided above, %-identity is: for sequence A being the sequence of the invention (6/9)*100=66.7%; for sequence B being the sequence of the invention (6/8)*100=75%.

The term “nucleic acid construct” as used herein refers to a nucleic acid molecule, either single- or double-stranded, which is isolated from a naturally occurring gene or is modified to contain segments of nucleic acids in a manner that would not otherwise exist in nature or is synthetic.

The term “nucleic acid construct” is synonymous with the term “expression cassette” when the nucleic acid construct contains the control sequences required for expression of a polynucleotide.

The term “control sequence” or “genetic control element” is defined herein to include all sequences affecting the expression of a polynucleotide, including but not limited thereto, the expression of a polynucleotide encoding a polypeptide. Each control sequence may be native or foreign to the polynucleotide or native or foreign to each other. Such control sequences include, but are not limited to, promoter sequence, 5′-UTR (also called leader sequence), ribosomal binding site (RBS), 3′-UTR, and transcription start and stop sites.

The term “functional linkage” or “operably linked” with respect to regulatory elements is to be understood as meaning the sequential arrangement of a regulatory element (including but not limited thereto a promoter) with a nucleic acid sequence to be expressed and, if appropriate, further regulatory elements (including but not limited thereto a terminator) in such a way that each of the regulatory elements can fulfil its intended function to allow, modify, facilitate or otherwise influence expression of said nucleic acid sequence. For example, a control sequence is placed at an appropriate position relative to the coding sequence of the polynucleotide sequence such that the control sequence directs the expression of the coding sequence of a polypeptide.

A “promoter” or “promoter sequence” is a nucleotide sequence located upstream of a gene on the same strand as the gene that enables that gene's transcription. A promoter is generally followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

As used herein, the term “isolated DNA molecule” refers to a DNA molecule at least partially separated from other molecules normally associated with it in its native or natural state. The term “isolated” preferably refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods well known to those skilled in the art can be used to isolate and manipulate a polynucleotide, or fragment thereof, as disclosed herein. For example, polymerase chain reaction (PCR) technology can be used to amplify a particular starting polynucleotide molecule and/or to produce variants of the original molecule. Polynucleotide molecules, or fragment thereof, can also be obtained by other techniques, such as by directly synthesizing the fragment by chemical means, as is commonly practiced by using an automated oligonucleotide synthesizer. A polynucleotide can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments of the method include those wherein the polynucleotide is at least one selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used.

As used herein, “recombinant” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if (a) that nucleotide sequence is present in a context other than its natural one, for example by virtue of being (i) cloned into any type of artificial nucleic acid vector or (ii) moved or copied to another location of the original genome, or if (b) the nucleotide sequence is mutagenized such that it differs from the wild type sequence. The term recombinant also can refer to an organism having a recombinant material, e.g., a plant that comprises a recombinant nucleic acid is a recombinant plant.

The term “transgenic” refers to an organism, preferably a plant or part thereof, or a nucleic acid that comprises a heterologous polynucleotide. Preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been so altered by the presence of heterologous nucleic acid including those transgenic organisms or cells initially so altered, as well as those created by crosses or asexual propagation from the initial transgenic organism or cell. A “recombinant” organism preferably is a “transgenic” organism. The term “transgenic” as used herein is not intended to encompass the alteration of the genome (chromosomal or extra-chromosomal) by conventional plant breeding methods (e.g., crosses) or by naturally occurring events such as, e.g., self-fertilization, random cross-fertilization, non-recombinant viral infection, non-recombinant bacterial transformation, non-recombinant transposition, or spontaneous mutation.

As used herein, “mutagenized” refers to an organism or nucleic acid thereof having alteration(s) in the biomolecular sequence of its native genetic material as compared to the sequence of the genetic material of a corresponding wildtype organism or nucleic acid, wherein the alteration(s) in genetic material were induced and/or selected by human action. Examples of human action that can be used to produce a mutagenized organism or DNA include, but are not limited to, treatment with a chemical mutagen such as EMS and subsequent selection with herbicide(s); or by treatment of plant cells with x-rays and subsequent selection with herbicide(s). Any method known in the art can be used to induce mutations. Methods of inducing mutations can induce mutations in random positions in the genetic material or can induce mutations in specific locations in the genetic material (i.e., can be directed mutagenesis techniques), such as by use of a genoplasty technique. In addition to unspecific mutations, according to the invention a nucleic acid can also be mutagenized by using mutagenesis means with a preference or even specificity for a particular site, thereby creating an artificially induced heritable allele according to the present invention. Such means, for example site specific nucleases, including for example zinc finger nucleases (ZFNs), meganucleases, transcription activator-like effector nucleases (TALENS) (Malzahn et al., Cell Biosci, 2017, 7:21) and clustered regularly interspaced short palindromic repeats/CRISPR-associated nuclease (CRISPR/Cas) with an engineered crRNA/tracr RNA (for example as a single-guide RNA, or as modified crRNA and tracrRNA molecules which form a dual molecule guide), and methods of using this nucleases to target known genomic locations, are well known in the art (see reviews by Bortesi and Fischer, 2015, Biotechnology Advances 33: 41-52; and by Chen and Gao, 2014, Plant Cell Rep 33: 575-583, and references within).

As used herein, a “genetically modified organism” (GMO) is an organism whose genetic characteristics contain alteration(s) that were produced by human effort causing transfection that results in transformation of a target organism with genetic material from another or “source” organism, or with synthetic or modified-native genetic material, or an organism that is a descendant thereof that retains the inserted genetic material. The source organism can be of a different type of organism (e.g., a GMO plant can contain bacterial genetic material) or from the same type of organism (e.g., a GMO plant can contain genetic material from another plant).

As used herein, “wildtype” or “corresponding wildtype plant” means the typical form of an organism or its genetic material, as it normally occurs, as distinguished from e.g. mutagenized and/or recombinant forms. Similarly, by “control cell”, “wildtype” “control plant, plant tissue, plant cell or host cell” is intended a plant, plant tissue, plant cell, or host cell, respectively, that lacks the particular polynucleotide of the invention that are disclosed herein. The use of the term “wildtype” is not, therefore, intended to imply that a plant, plant tissue, plant cell, or other host cell lacks recombinant DNA in its genome, and/or does not possess fungal resistance characteristics that are different from those disclosed herein.

As used herein, “descendant” refers to any generation plant. A progeny or descendant plant can be from any filial generation, e.g., F1, F2, F3, F4, F5, F6, F7, etc. In some embodiments, a descendant or progeny plant is a first, second, third, fourth, fifth, sixth, seventh, eighth, ninth, or tenth generation plant.

The term “plant” is used herein in its broadest sense and pertains to plants in all stages of maturity and regardless of their generation method, including leaf-bearing callus cultures, germinated seed, shoots, plants with 1 or more canopy layers, flowering plants, inseminated plants and seed pod bearing plants. Furthermore, the term “plant” is used herein as a shorthand not differentiating between whole plants and parts thereof, including cell or tissue cultures, plant components or organs (e.g., leaves, stems, roots, etc.), plant tissues, seeds, plant cells, and/or progeny of the same. A “plant cell” is a biological cell of a plant, taken from a plant or derived through culture from a cell taken from a plant.

The invention particularly applies to plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato or tobacco. The plant preferably is not of taxonomic family Solanaceae, more preferably not of sub-family Solanoidae.

Most preferably the plant is a soybean plant. The term “soybean” means members of the taxonomic species Glycine max and includes all plants that permit breeding; they can be crossed with a member of said species to produce viable offspring, preferably fertile offspring. The term “soybean” in particular includes perennial and annual members of genus Glycine, wild soybean species, plants of taxonomic species Glycine clandestina, Glycine falcata, Glycine tabacina and Glycine tomentella as well as those plants belonging to taxonomic species Glycine gracilis or Glycine soja that permit breeding between species.

As plants are grown by cell division, references herein to plants comprising the Pti5, CaSAR and/or Pti5-CaSAR fusion proteins and corresponding genes always also refer to (1) one or more cells comprising a nucleic acid coding for said genes and (2) to plant parts, in particular organs, preferably leaves, of such plants comprising such cells. The term “soybean” thus also encompasses polyploid plants and cells thereof.

The invention provides a method for conferring, modifying or increasing resistance against multiple pathogen infections of a plant, plant part, or plant cell in comparison to a respective wild type plant, wild type plant part, or wild type plant cell. Due to the broad applicability of the invention simultaneously against several pathogens of different infection biology, the invention also provides a method for conferring, modifying or increasing resistance against any single of those pathogens. This is expressed herein by the phrase “one or multiple pathogen infections”, “one or multiple pathogens”, “single or multiple pathogen pressure” and the like.

The pathogens comprise members of the following taxa: (1) order Pleosporales, preferably family Corynesporascaceae, (2) order Mycosphaerellales, preferably family Mycosphaerellaceae, (3) order Erysiphales, preferably family Erysiphaceae, (4) order Botryosphaeriales, preferably family Phyllostictaceae. In addition to or alternatively to the definition of the previous sentence, the pathogens may also comprise the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot.

The method comprises the step of increasing the production and/or accumulation of (1) a combination of at least a Pti5 protein and a CaSAR protein, (2) a combination of a Pti5 protein and a Pti5-CaSAR fusion protein, or a combination of a CaSAR protein and a Pti5-CaSAR fusion protein, or (3) a Pti5-CaSAR fusion protein in the plant, plant part or plant cell.

Plants comprising either a Pti5 or CaSAR gene have been described before, among others, in WO2013001435 and WO2014076614. The documents describe that such plants are resistant against Asian soybean rust (Phakopsora pachyrhizi). However, they do not show any simultaneous increase in resistance against multiple pathogens and diseases as discussed herein, i.e.
the pathogens of the following taxa: (1) order Pleosporales, preferably family Corynesporascaceae, (2) order Mycosphaerellales, preferably family Mycosphaerellaceae, (3) order Erysiphales, preferably family Erysiphaceae, (4) order Botryosphaeriales, preferably family Phyllostictaceae and/or
the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot.

Furthermore, it was surprising that a combined presence of a CaSAR protein and a Pti5 protein, or such respective domains of a fusion protein, in pants, in particular in soybean, leads to a broad spectrum resistance against many pathogens with different lifestyles, for example biotrophic powdery mildew, hemibiotrophic Septoria glycines, heminecrotrophic Phakopsora pachyrhizi and necrotrophic Corynespora cassiicola.

Correspondingly, the invention provides a method for conferring, modifying or increasing resistance against multiple pathogen infections of a plant, plant part, or plant cell in comparison to a respective wild type plant, wild type plant part, or wild type plant cell, wherein the resistance is simultaneously against biotrophic and hemibiotrophic pathogens, biotrophic and necrotrophic pathogens, hemibiotrophic pathogens and necrotrophic pathogens or against biotrophic, hemibiotrophic and necrotrophic pathogens. More preferably, the invention provides such simultaneous resistance against 1) order Pleosporales, preferably family Corynesporascaceae and 2) order Mycosphaerellales, preferably family Mycosphaerellaceae; 1) order Pleosporales, preferably family Corynesporascaceae and 2) order Erysiphales, preferably family Erysiphaceae; 1) order Pleosporales, preferably family Corynesporascaceae and 2) order Botryosphaeriales, preferably family Phyllostictaceae; 1) order Mycosphaerellales, preferably family

Mycosphaerellaceae and 2) order Erysiphales, preferably family Erysiphaceae; 1) order Mycosphaerellales, preferably family Mycosphaerellaceae and 2) order Botryosphaeriales, preferably family Phyllostictaceae; and/or 1) order Erysiphales, preferably family Erysiphaceae and 2) order Botryosphaeriales, preferably family Phyllostictaceae.

Most preferably the invention provides a method for conferring, modifying or increasing resistance against pathogen infections of a plant, plant part, or plant cell in comparison to a respective wild type plant, wild type plant part, or wild type plant cell, wherein the resistance is effective simultaneously against all pathogens of
order Pleosporales, preferably family Corynesporascaceae,
order Mycosphaerellales, preferably family Mycosphaerellaceae,
order Erysiphales, preferably family Erysiphaceae, and
order Botryosphaeriales, preferably family Phyllostictaceae.

Preferably according to the invention, the pathogen of family Corynesporascaceae is a member of genus Corynespora, more preferably of any of the species Corynespora cambrensis, Corynespora cassiicola, Corynespora citricola, Corynespora doipuiensis, Corynespora encephalarti, Corynespora lignicola, Corynespora ligustri, Corynespora olivacea, Corynespora proliferata, Corynespora pseudocassiicola, Corynespora smithii, Corynespora submersa, Corynespora thailandica or Corynespora torulosa. Most preferably the Pti5-CaSAR combination of the present invention is applied to reduce infections by Corynespora cassiicola.

Further preferably according to the invention, the pathogen of family Mycosphaerellaceae is a member of genus Septoria, more preferably of any of the species Septoria empetri, Septoria genistae, Septoria martiniana, Septoria donacis, Septoria abei, Septoria aciculosa, Septoria aegopodina, Septoria agrimoniicola, Septoria albopunctata, Septoria allii, Septoria alni, Septoria alnifolia, Septoria anaxaea, Septoria anthrisci, Septoria anthurii, Septoria apiicola, Septoria apocyni, Septoria artemisiae, Septoria arundinacea, Septoria astericola, Septoria atropurpurea, Septoria bothriospermi, Septoria bupleuri, Septoria bupleuricola, Septoria calendulae, Septoria callistephi, Septoria campanulae, Septoria canadensis, Septoria cannabis, Septoria carvi, Septoria celastri, Septoria cerastii, Septoria chamaecysti, Septoria chelidonii, Septoria chromolaenae, Septoria chrysanthemella, Septoria cirsii, Septoria citri, Septoria citricola, Septoria clematidis, Septoria codonopsidis, Septoria colensoi, Septoria convolvuli, Septoria coprosmae, Septoria crepidis, Septoria cretae, Septoria cruciatae, Septoria cucubali, Septoria cucurbitacearum, Septoria cytisi, Septoria dearnessii, Septoria digitalis, Septoria dispori, Septoria dolichospora, Septoria dysentericae, Septoria eclipticola, Septoria ekmaniana, Septoria epambrosiae, Septoria epilobii, Septoria erigerontis, Septoria escalloniae, Septoria eucalyptorum, Septoria exotica, Septoria firouraghina, Septoria floridae, Septoria galeopsidis, Septoria gaurina, Septoria gentianae, Septoria gerberae, Septoria gladioli, Septoria glycines, Septoria glycinicola, Septoria guilanensis, Septoria helianthi, Septoria helianthicola, Septoria hibiscicola, Septoria hippocastani, Septoria justiciae, Septoria lactucae, Septoria lamii, Septoria lamiicola, Septoria lavandulae, Septoria lepidii, Septoria lepidiicola, Septoria leptostachya, Septoria leucanthemi, Septoria limonum, Septoria linicola, Septoria longipes, Septoria lycoctoni, Septoria lycopersici (tomato leaf spot fungus), Septoria lycopicola, Septoria lysimachiae, Septoria macropoda, Septoria malagutii, Septoria matricariae, Septoria matthiolae, Septoria mazi, Septoria melissae, Septoria menthae, Septoria microspora, Septoria napelli, Septoria obesa, Septoria oenanthicola, Septoria oenanthis, Septoria orchidearum, Septoria ostryae, Septoria oudemansii, Septoria pachyspora, Septoria paridis, Septoria passiflorae, Septoria passifloricola, Septoria perillae, Septoria petroselini, Septoria phalaridis, Septoria phlogis, Septoria pileicola, Septoria pistaciae, Septoria pistaciarum, Septoria polygonorum, Septoria posoniensis, Septoria protearum, Septoria provencialis, Septoria pseudonapelli, Septoria putrida, Septoria robiniae, Septoria rosae, Septoria rudbeckiae, Septoria rumicum, Septoria saccardoi, Septoria sambucina, Septoria sanguisorbigena, Septoria saposhnikoviae, Septoria scabiosicola, Septoria schnabliana, Septoria senecionis, Septoria sigesbeckiae, Septoria sii, Septoria sisyrinchii, Septoria sonchi, Septoria stachydicola, Septoria stachydis, Septoria stellariae, Septoria steviae, Septoria taleshana, Septoria tanaceti, Septoria taraxaci, Septoria tatarica, Septoria tinctoriae, Septoria tormentillae, Septoria urticae, Septoria verbascicola, Septoria verbenae, Septoria villarsiae, Septoria violae-palustris, Septoria violae-patrinii, Septoria pini-thunbergii, Septoria azaleae, Septoria berberidis, Septoria betulae, Septoria gei, Septoria hyperici, Septoria menispermi, Septoria musiva, Septoria patriniae, Septoria populicola, Septoria halophila or Septoria passerinii, or is a member of genus Cercospora, more preferably of any of the species Cercospora acaciae-mangii, Cercospora achyranthis, Cercospora agavicola, Cercospora alchemillicola, Cercospora althaeina, Cercospora alyssopsidis, Cercospora americana, Cercospora apii, Cercospora apiicola, Cercospora arctii-ambrosiae, Cercospora arecacearum, Cercospora ariminensis, Cercospora armoraciae, Cercospora artemisiae, Cercospora asparagi, Cercospora aurantia, Cercospora balsaminiana, Cercospora beninensis, Cercospora berteroae, Cercospora beticola, Cercospora bidentis, Cercospora bizzozeriana, Cercospora brachiata, Cercospora brassicicola, Cercospora broussonetiae, Cercospora campi-silii, Cercospora canescens, Cercospora capsici, Cercospora capsicigena, Cercospora caricis, Cercospora carotae, Cercospora celosiae, Cercospora chenopodii, Cercospora chinensis, Cercospora christellae, Cercospora chrysanthemi, Cercospora chrysanthemoides, Cercospora citrullina, Cercospora cocciniae, Cercospora codiaei, Cercospora coniogrammes, Cercospora convolvulicola, Cercospora conyzae-canadensis, Cercospora corchori, Cercospora cylindracea, Cercospora cypericola, Cercospora cyperina, Cercospora delaireae, Cercospora dianellicola, Cercospora dichondrae, Cercospora dioscoreae-pyrifoliae, Cercospora dispori, Cercospora eremochloae, Cercospora erysimi, Cercospora eucommiae, Cercospora euphorbiae-sieboldianae, Cercospora fagopyri, Cercospora flagellaris, Cercospora fukushiana, Cercospora gamsiana, Cercospora gerberae, Cercospora glycinicola, Cercospora gomphrenigena, Cercospora gossypii, Cercospora guatemalensis, Cercospora hawaiiensis, Cercospora hayi, Cercospora helianthicola, Cercospora hydrangeae, Cercospora ipomoeae, Cercospora ipomoeae-pedis-caprae, Cercospora iranica, Cercospora ischaemi, Cercospora janseana, Cercospora jatrophiphila, Cercospora kalmiae, Cercospora kikuchii, Cercospora lactucae-sativae, Cercospora lagenariae, Cercospora longissima, Cercospora loranthi, Cercospora maculicola, Cercospora malayensis, Cercospora malloti, Cercospora malvacearum, Cercospora manihobae, Cercospora manoa, Cercospora meliicola, Cercospora mercurialis, Cercospora mikaniae, Cercospora mikaniicola, Cercospora modiolae, Cercospora musigena, Cercospora nasturtii, Cercospora neomaricae, Cercospora nicotianae, Cercospora olivascens, Cercospora oroxyli, Cercospora parakouensis, Cercospora penzigii, Cercospora phaseoli-lunati, Cercospora physalidis, Cercospora piaropi, Cercospora pileicola, Cercospora pisi-sativi, Cercospora plantaginis, Cercospora poae, Cercospora polygonacea, Cercospora populicola, Cercospora pseudochenopodii, Cercospora pseudokalanchoes, Cercospora punctiformis, Cercospora rautensis, Cercospora resedae, Cercospora rhynchophora, Cercospora ricinella, Cercospora rodmanii, Cercospora rumicis, Cercospora samambaiae, Cercospora senecionis-walkeri, Cercospora sesami, Cercospora setariae, Cercospora sidicola, Cercospora sojina, Cercospora solani, Cercospora solani-betacei, Cercospora sophorae, Cercospora sorghi, Cercospora sorghicola, Cercospora subulata, Cercospora tagetea, Cercospora tecta, Cercospora tentaculifera, Cercospora tetragoniae, Cercospora tezpurensis, Cercospora uwebrauniana, Cercospora vignae-subterraneae, Cercospora vignicola, Cercospora vignigena, Cercospora violae, Cercospora viticola, Cercospora zeae-maydis, Cercospora zebrina, Cercospora zeina, Cercospora zinniae, Cercospora zinniicola, Cercospora zonata or Cercospora zorniicola. Most preferably the Pti5-CaSAR combination of the present invention is applied to reduce infections by Septoria glycines, Cercospora kikuchii and/or Cercospora sojina.

Further preferably according to the invention, the pathogen of family Erysiphaceae is a member of genus Arthrocladiella, Blumeria, Brasiliomyces, Bulbomicroidium, Caespitotheca, Cystotheca, Erysiphe, Euoidium, Fibroidium, Golovinomyces, Leveillula, Microidium, Microsphaera, Neoerysiphe, Oidiopsis, Oidium, Ovulariopsis, Parauncinula, Phyllactinia, Pleochaeta, Podosphaera, Pseudoidium, Salmonomyces, Sawadaea, Setoidium, Striatoidium or Takamatsuella, even more preferably of genus Erysiphe, even more preferably of any of the species Erysiphe abbreviata, Erysiphe abeliicola, Erysiphe acaenae, Erysiphe acantholimonis, Erysiphe actinidiae, Erysiphe adunca, Erysiphe akebiae, Erysiphe alphitoides, Erysiphe aphananthes, Erysiphe aquilegiae, Erysiphe arcuata, Erysiphe asclepiadis, Erysiphe asiatica, Erysiphe astragali, Erysiphe aucubae, Erysiphe australiana, Erysiphe azaleae, Erysiphe azerbaijanica, Erysiphe baeumleri, Erysiphe balbisiae, Erysiphe baliensis, Erysiphe baptisiae, Erysiphe begoniae, Erysiphe begoniicola, Erysiphe berberidicola, Erysiphe berberidis, Erysiphe berchemiae, Erysiphe betae, Erysiphe betulina, Erysiphe blasti, Erysiphe bremeri, Erysiphe buhrii, Erysiphe bulbouncinula, Erysiphe bunkiniana, Erysiphe capreae, Erysiphe caricae, Erysiphe caricae-papayae, Erysiphe carpini-cordatae, Erysiphe carpini-laxiflorae, Erysiphe carpinicola, Erysiphe carpophila, Erysiphe castaneigena, Erysiphe catalpae, Erysiphe celtidis, Erysiphe chifengensis, Erysiphe chloranthi, Erysiphe circaeae, Erysiphe clandestina, Erysiphe clethrae, Erysiphe convolvuli, Erysiphe coriariae, Erysiphe coriariigena, Erysiphe cornicola, Erysiphe cornutae, Erysiphe corylacearum, Erysiphe coryli-americanae, Erysiphe corylicola, Erysiphe corylopsidis, Erysiphe cruciferarum, Erysiphe densa, Erysiphe deutziae, Erysiphe deutziicola, Erysiphe diervillae, Erysiphe diffusa (also known as Microsphaera diffusa), Erysiphe digitata, Erysiphe dimorpha, Erysiphe discariae, Erysiphe divaricata, Erysiphe ehrenbergii, Erysiphe elevata, Erysiphe ellisii, Erysiphe epigena, Erysiphe epimedii, Erysiphe erlangshanensis, Erysiphe eschscholziae, Erysiphe euonymi, Erysiphe euonymi-japonici, Erysiphe euonymicola, Erysiphe euphorbiae, Erysiphe fallax, Erysiphe fernandoae, Erysiphe fimbriata, Erysiphe flexibilis, Erysiphe flexuosa, Erysiphe fraxinea, Erysiphe fraxinicola, Erysiphe frickii, Erysiphe friesii, Erysiphe galii, Erysiphe glycines, Erysiphe gracilis, Erysiphe guarinonii, Erysiphe havrylenkoana, Erysiphe hedwigii, Erysiphe helwingiae, Erysiphe heraclei, Erysiphe hiratae, Erysiphe hommae, Erysiphe howeana, Erysiphe huayinensis, Erysiphe hydrangeae, Erysiphe hyperici, Erysiphe hypogena, Erysiphe hypophylla, Erysiphe intermedia, Erysiphe ipomoeae, Erysiphe izuensis, Erysiphe japonica, Erysiphe javanica, Erysiphe juglandis, Erysiphe katumotoi, Erysiphe kenjiana, Erysiphe kissiana, Erysiphe knautiae, Erysiphe kusanoi, Erysiphe lespedezae, Erysiphe ligustri, Erysiphe limonii, Erysiphe linderae, Erysiphe liquidambaris, Erysiphe liriodendri, Erysiphe ljubarskii, Erysiphe longiappendiculata, Erysiphe longifilamentosa, Erysiphe longissima, Erysiphe lonicerae, Erysiphe lonicerina, Erysiphe ludens, Erysiphe lupini, Erysiphe lycopsidis, Erysiphe lythri, Erysiphe machiliana, Erysiphe macleayae, Erysiphe magellanica, Erysiphe magnifica, Erysiphe magnoliae, Erysiphe magnoliicola, Erysiphe magnusii, Erysiphe malvae, Erysiphe mandshurica, Erysiphe manihoticola, Erysiphe mayorii, Erysiphe medicaginis, Erysiphe menispermi, Erysiphe michikoae, Erysiphe miranda, Erysiphe miurae, Erysiphe miyabei, Erysiphe monascogera, Erysiphe monoperidiata, Erysiphe mori, Erysiphe multappendicis, Erysiphe myoschili, Erysiphe myzodendri, Erysiphe necator (grape powdery mildew), Erysiphe neolycopersici, Erysiphe nishidana, Erysiphe nomurae, Erysiphe nothofagi, Erysiphe oehrensii, Erysiphe oleosa, Erysiphe orixae, Erysiphe ornata, Erysiphe ostryae, Erysiphe ovidiae, Erysiphe paeoniae, Erysiphe pakistanica, Erysiphe palczewskii, Erysiphe panacis, Erysiphe paracarpinicola, Erysiphe paradoxa, Erysiphe patagoniaca, Erysiphe penicillata, Erysiphe peruviana, Erysiphe phyllanthi, Erysiphe pileae, Erysiphe pisi, Erysiphe platani, Erysiphe polygoni, Erysiphe prunastri, Erysiphe pseudocarpinicola, Erysiphe pseudocorylacearum, Erysiphe pseudogracilis, Erysiphe pseudolonicerae, Erysiphe pseudoregularis, Erysiphe pseudoviburni, Erysiphe pulchra, Erysiphe punicae, Erysiphe quercicola, Erysiphe rayssiae, Erysiphe rhamnicola, Erysiphe ribicola, Erysiphe robiniae, Erysiphe robiniicola, Erysiphe rodgersiae, Erysiphe rosae, Erysiphe russellii, Erysiphe ruyongzhengiana, Erysiphe salicicola, Erysiphe salicis, Erysiphe salmonii, Erysiphe schizandrae, Erysiphe schizophragmatis, Erysiphe sedi, Erysiphe sengokui, Erysiphe sesbaniae, Erysiphe sidae, Erysiphe simulans, Erysiphe sinomenii, Erysiphe staphyleae, Erysiphe symphoricarpi, Erysiphe syringae, Erysiphe syringae-japonicae, Erysiphe takamatsui, Erysiphe tectonae, Erysiphe thaxteri, Erysiphe thermopsidis, Erysiphe thesii, Erysiphe thuemenii, Erysiphe togashiana, Erysiphe tortilis, Erysiphe trifolii, Erysiphe trifoliorum, Erysiphe trina, Erysiphe ulmariae, Erysiphe ulmi, Erysiphe uncinuloides, Erysiphe urticae, Erysiphe vaccinii, Erysiphe vanbruntiana, Erysiphe verniciferae, Erysiphe viburni, Erysiphe viburni-plicati, Erysiphe viburniphila, Erysiphe viciae-unijugae, Erysiphe vignae, Erysiphe wadae, Erysiphe wallrothii, Erysiphe weigelae or Erysiphe zelkowae. Most preferably the Pti5-CaSAR combination of the present invention is applied to reduce infections by Erysiphe diffusa.

And preferably according to the invention, the pathogen of family Phyllostictaceae is a member of genus Phyllosticta, more preferably of any of the species Phyllosticta abieticola, Phyllosticta abietis, Phyllosticta acaciigena, Phyllosticta alcides, Phyllosticta alliacea, Phyllosticta aloeicola, Phyllosticta ampelicida, Phyllosticta ardisiicola, Phyllosticta aristolochiicola, Phyllosticta artocarpina, Phyllosticta aspidistricola, Phyllosticta aucubae-japonicae, Phyllosticta austroafricana, Phyllosticta azevinhi, Phyllosticta beaumarisii, Phyllosticta bifrenariae, Phyllosticta braziliana, Phyllosticta camelliae, Phyllosticta capitalensis, Phyllosticta cf. capitalensis G27, Phyllosticta cf. capitalensis G9, Phyllosticta caprifolii, Phyllosticta carissicola, Phyllosticta carochlae, Phyllosticta catimbauensis, Phyllosticta cavendishii, Phyllosticta cirsii, Phyllosticta citriasiana, Phyllosticta citribraziliensis, Phyllosticta citricarpa, Phyllosticta citrichinaensis, Phyllosticta citrimaxima, Phyllosticta citrullina, Phyllosticta concentrica, Phyllosticta cordylinophila, Phyllosticta coryli, Phyllosticta cruenta, Phyllosticta cryptomeriae, Phyllosticta cussoniae, Phyllosticta elongata, Phyllosticta encephalarticola, Phyllosticta ericarum, Phyllosticta eugeniae, Phyllosticta fallopiae, Phyllosticta flevolandica, Phyllosticta foliorum, Phyllosticta gardeniicola, Phyllosticta hagahagaensis, Phyllosticta hakeicola, Phyllosticta hamamelidis, Phyllosticta heveae, Phyllosticta hostae, Phyllosticta hubeiensis, Phyllosticta hymenocallidicola, Phyllosticta hypoglossi, Phyllosticta ilicis-aquifolii, Phyllosticta illicii, Phyllosticta iridigena, Phyllosticta jasmini, Phyllosticta juglandis, Phyllosticta kerriae, Phyllosticta kobus, Phyllosticta lauridiae, Phyllosticta leucothoicola, Phyllosticta ligustri, Phyllosticta ligustricola, Phyllosticta longicauda, Phyllosticta maculata, Phyllosticta mangiferae-indicae, Phyllosticta mimusopisicola, Phyllosticta minima, Phyllosticta miurae, Phyllosticta musaechinensis, Phyllosticta musarum, Phyllosticta musicola, Phyllosticta neopyrolae, Phyllosticta ophiopogonis, Phyllosticta owaniana, Phyllosticta pachysandricola, Phyllosticta papayae, Phyllosticta paracapitalensis, Phyllosticta paracitricarpa, Phyllosticta parthenocissi, Phyllosticta partricuspidatae, Phyllosticta paxistimae, Phyllosticta persooniae, Phyllosticta phaseolina, Phyllosticta philoprina, Phyllosticta phoenicis, Phyllosticta pilospora, Phyllosticta podocarpi, Phyllosticta podocarpicola, Phyllosticta populina, Phyllosticta pseudotsugae, Phyllosticta pyrolae, Phyllosticta rhizophorae, Phyllosticta rubella, Phyllosticta schimae, Phyllosticta schimicola, Phyllosticta sojicola, Phyllosticta speewahensis, Phyllosticta sphaeropsoidea, Phyllosticta spinarum, Phyllosticta styracicola, Phyllosticta telopeae, Phyllosticta vaccinii, Phyllosticta vacciniicola, Phyllosticta vitis-rotundifoliae, Phyllosticta westeae, Phyllosticta yuccae or Phyllosticta yugokwa. Most preferably the Pti5-CaSAR combination of the present invention is applied to reduce infections by Phyllosticta sojicola.

Thus, preferably the method or use of the present invention confers, modifies or increases resistance against two or more species selected from the group of Corynespora cassiicola, Septoria glycines, Cercospora kikuchii, Cercospora sojina, Fusarium virguliforme and Phyllosticta sojicola, preferably three or more, more preferably against all of said species.

According to the present invention, the gene coding for a Pti5 protein codes for a Pti5 protein which has at least 72%, preferably at least 74%, more preferably at least 78%, more preferably at least 79%, more preferably at least 83%, more preferably at least 99%, more preferably 100% sequence identity to SEQ ID NO. 1. As shown in the examples, particularly good results have been obtained with a Pti5 gene coding for the protein of SEQ ID NO. 1. Also preferred are, however, homologs of such Pti5 protein which exhibit less than 100% sequence identity to SEQ ID NO. 1, i.e. proteins whose amino acid sequence does not consist of the sequence according to SEQ ID NO. 1. Such Pti5 proteins are, in decreasing order of preference and decreasing sequence identity to SEQ ID NO. 1:

| | % sequence identity | % sequence similarity |
|---|---|---|
| Uniprot identifier | to SEQ ID NO. 1 | to SEQ ID NO. 1 |
| PTI5_SOLLC | 99.4 | 100 |
| M1AQ94_SOLTU | 83 | 85.7 |
| A0A2G3A6U8_CAPAN | 79.1 | 84.4 |
| A0A2G2XEI7_CAPBA | 78.6 | 83.9 |
| A0A2G3D5K5_CAPCH | 78.6 | 83.9 |
| A0A1S4BF73_TOBAC | 74.6 | 80.4 |
| A0A1U7WC00_NICSY | 74.6 | 80.4 |
| A0A1S4A5G9_TOBAC | 72.4 | 79.9 |

According to the present invention, the gene coding for a CaSAR protein codes for a CaSAR protein which has at least 72%, preferably at least 74%, more preferably at least 78%, more preferably at least 79%, more preferably at least 83%, more preferably at least 99%, more preferably 100% sequence identity to SEQ ID NO. 2. The sequence according to SEQ ID NO. 2 has also been published under Uniprot identifier Q8W2C1_CAPAN. As shown in the examples, particularly good results have been obtained with a CaSAR gene coding for the protein of SEQ ID NO. 2. Also preferred are, however, homologs of such CaSAR protein which exhibit less than 100% sequence identity to SEQ ID NO. 2, i.e. proteins whose amino acid sequence does not consist of the sequence according to SEQ ID NO. 2. Such CaSAR proteins are, in decreasing order of preference and decreasing sequence identity to SEQ ID NO. 2:

| | % sequence identity | % sequence similarity |
|---|---|---|
| Uniprot identifier | to SEQ ID NO. 2 | to SEQ ID NO. 2 |
| A0A2G2X990_CAPBA | 96.5 | 98.8 |
| Q947G5_CAPAN | 95.3 | 97.7 |
| A0A2G2X9U8_CAPBA | 95.3 | 96.5 |
| A0A2G3CEJ1_CAPCH | 91.9 | 96.5 |
| A0A2G2X931_CAPBA | 89.5 | 93 |
| M1BEK3_SOLTU | 68.5 | 77.5 |
| A0A3Q7J4M2_SOLLC | 61.8 | 77.5 |
| A0A2G3CRF6_CAPCH | 61.1 | 75.6 |
| A0A2G2ZTB6_CAPAN | 61.1 | 75.6 |
| A0A2G2W296_CAPBA | 61.1 | 75.6 |
| A0A2G2WZ87_CAPBA | 60 | 76.7 |

Furthermore, the Pti5-CaSAR fusion protein, if present, comprises a Pti5 protein domain linked, via a linker, to a CaSAR protein domain. The Pti5 domain is a protein domain which has at least 72%, preferably at least 74%, more preferably at least 78%, more preferably at least 79%, more preferably at least 83%, more preferably at least 99%, more preferably 100% sequence identity to SEQ ID NO. 1, or comprises or consists of a protein according to any of the above indicated Uniprot homologs of Pti5. The CaSAR domain is a protein domain which has at least 72%, preferably at least 74%, more preferably at least 78%, more preferably at least 79%, more preferably at least 83%, more preferably at least 99%, more preferably 100% sequence identity to SEQ ID NO. 2, or comprises or consists of a protein according to any of the above indicated Uniprot homologs of CaSAR.

The method of the present invention preferably further comprises the steps of
i) stably transforming a plant cell with
   a) a heterologous expression cassette comprising a Pti5 gene and, optionally in a separate transformation step, with a heterologous expression cassette comprising a CaSAR gene, and/or
   b) a heterologous expression cassette comprising a Pti5-CaSAR fusion gene,
ii) regenerating a plant from the plant cell, and
iii) expressing the respective Pti5, CaSAR and/or Pti5-CaSAR fusion proteins in cells of said plant.

As described herein, such transformation can be performed using standard laboratory methods and materials. It is a particular advantage of the present invention that imparting broad disease resistance against pathogens of strongly different modes of pathogenicity can be performed using such tried and trusted techniques.

In the method of the present invention, the plant preferably comprises a heterologous Pti5 expression cassette and a heterologous CaSAR expression cassette, wherein for each expression cassette the respective Pti5 or CaSAR gene is operably linked to any of
a) a constitutively active promoter,
b) a tissue-specific or tissue-preferred promoter,
c) a promoter inducible by exposition of the plant to a pest, preferably a fungal pest.

Where the promoter is inducible by exposition of the plant to a fungal pest, the fungal pest is preferably any of the pathogens of
the following taxa: (1) order Pleosporales, preferably family Corynesporascaceae, (2) order Mycosphaerellales, preferably family Mycosphaerellaceae, (3) order Erysiphales, preferably family Erysiphaceae, (4) order Botryosphaeriales, preferably family Phyllostictaceae and/or
the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot.

Thus, the expression of the Pti5 and CaSAR proteins, and/or the expression of the Pti5-CaSAR fusion protein, is preferably induced or upregulated upon exposure of a cell of the plant to at least one of the aforementioned fungal pathogens. Thus, exposure to the pathogen leads to emergence or increase of resistance against the very pathogen.

The invention also provides a method of producing a crop plant showing improved resistance against multiple pathogens compared to the corresponding wild type, comprising the steps of
i) crossing two different crop plants, wherein at least one of the plants comprises a Pti5 gene and a CaSAR gene, and/or a Pti5-CaSAR fusion gene, to produce members of a filial generation,
ii) detecting the presence of a polynucleotide indicative of the Pti5, CaSAR and/or Pti5-CaSAR fusion protein and in said members of the filial generation, and
ii) selecting at least one member of the filial generation with confirmed presence of the polynucleotide indicative of the Pti5, CaSAR and/or Pti5-CaSAR fusion protein,
   wherein the pathogens are selected from
   the following taxa: (1) order Pleosporales, preferably family Corynesporascaceae, (2) order Mycosphaerellales, preferably family Mycosphaerellaceae, (3) order Erysiphales, preferably family Erysiphaceae, (4) order Botryosphaeriales, preferably family Phyllostictaceae and/or
   the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot.

Crossing and selecting are old, reliable and standard techniques and allow for the rapid transfer of the broad pathogen resistance conferred by the present invention to diverse plants and plant varieties.

The invention also provides a farming method, i.e. a method of reducing or preventing multiple pathogen pressure of a field, comprising the step of cultivating, on said field, plants comprising
a) a heterologous expression cassette comprising a Pti5 gene and a heterologous expression cassette comprising a CaSAR gene, and/or
b) a heterologous expression cassette comprising a Pti5-CaSAR fusion gene, wherein the pathogens are selected from
   the following taxa: (1) order Pleosporales, preferably family Corynesporascaceae, (2) order Mycosphaerellales, preferably family Mycosphaerellaceae, (3) order Erysiphales, preferably family Erysiphaceae, (4) order Botryosphaeriales, preferably family Phyllostictaceae and/or
   the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot.

As described herein, plants expressing the Pti5 and CaSAR proteins of the present invention, optionally in combination with or replaced by a Pti5-CaSAR fusion protein as described according to the present invention, are less susceptible to the aforementioned pathogens than corresponding wild type plants. Thus, the plants according to the present invention provide less nutrition and support to the development of severe infestations by said pathogens. It is thus a particularly noteworthy advantage of the present invention that a farming method as described above not only reduces pressure of said pathogens on the field on which the plants according to the invention are cultivated, but also reduce pathogen pressure on neighbouring or adjacent fields.

According to the invention, the plant is a crop plant, preferably a dicotyledon, more preferably a plant of order Fabales, more preferably a plant of family Fabaceae, more preferably a plant of tribus Phaseoleae, more preferably of genus Amphicarpaea, Cajanus, Canavalia, Dioclea, Erythrina, Glycine, Arachis, Lathyrus, Lens, Pisum, Vicia, Vigna, Phaseolus or Psophocarpus, even more preferably of species Amphicarpaea bracteata, Cajanus cajan, Canavalia brasiliensis, Canavalia ensiformis, Canavalia gladiata, Dioclea grandiflora, Erythrina latissima, Phaseolus acutifolius, Phaseolus lunatus, Phaseolus maculatus, Psophocarpus tetragonolobus, Vigna angularis, Vigna mungo, Vigna unguiculata, Glycine albicans, Glycine aphyonota, Glycine arenaria, Glycine argyrea, Glycine canescens, Glycine clandestina, Glycine curvata, Glycine cyrtoloba, Glycine dolichocarpa, Glycine falcata, Glycine gracei, Glycine hirticaulis, Glycine lactovirens, Glycine latifolia, Glycine latrobeana, Glycine microphylla, Glycine peratosa, Glycine pindanica, Glycine pullenii, Glycine rubiginosa, Glycine stenophita, Glycine syndetika, Glycine tabacina, Glycine tomentella, Glycine gracilis, Glycine max, Glycine max x Glycine soja, Glycine soja, more preferably of species Glycine gracilis, Glycine max, Glycine max x Glycine soja, Glycine soja, most preferably of species Glycine max.

And the invention provides the use of (i) a combination of a Pti5 protein and a CaSAR protein, (ii) a Pti5-CaSAR fusion protein or (iii) a combination of a Pti5 or CaSAR protein and a Pti5-CaSAR fusion protein for conferring, modifying or increasing resistance against one or multiple pathogen infections of a plant, plant part, or plant cell in comparison to a respective wild type plant, wild type plant part, or wild type plant cell, wherein the pathogens are selected from
the following taxa: (1) order Pleosporales, preferably family Corynesporascaceae, (2) order Mycosphaerellales, preferably family Mycosphaerellaceae, (3) order Erysiphales, preferably family Erysiphaceae, (4) order Botryosphaeriales, preferably family Phyllostictaceae and/or
the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot.

It was a surprising advantage that the expression of merely two proteins, i.e. Pti5 and CaSAR, or the expression of a Pti5-CaSAR fusion protein (optionally with any of Pti5 or CaSAR proteins in addition), provides broad pathogen resistance.

The invention is described in further detail in the following, non-limiting examples.

### EXAMPLES

### Example 1: General Methods

The chemical synthesis of oligonucleotides can be affected, for example, in the known fashion using the phosphoamidite method (Voet, Voet, 2nd Edition, Wiley Press New York, pages 896-897). The cloning steps carried out for the purposes of the present invention such as, for example, restriction cleavages, agarose gel electrophoresis, purification of DNA fragments, transfer of nucleic acids to nitrocellulose and nylon membranes, linking DNA fragments, transformation of E. coli cells, bacterial cultures, phage multiplication and sequence analysis of recombinant DNA, are carried out as described by Sambrook et al., Cold Spring Harbor Laboratory Press (1989), ISBN 0-87969-309-6. The sequencing of recombinant DNA molecules is carried out with an MWG-Licor laser fluorescence DNA sequencer following the method of Sanger (Sanger et al., Proc. Natl. Acad. Sci. USA 74, 5463 (1977)).

### Example 2: Cloning of Vector to Express Pti5 and CaSAR Gene

A BiBAC (Binary Bacterial Artificial Chromosome) vector containing the combination of the expression cassettes p-PcUbi4-2::CaSAR_Gm::t-StCAT and p-PcUbi4-2::Pti5_V84I::t-StCAT was used. In addition a gene providing strong tolerance against PPO inhibitor herbicides, p-PcUbi4-2::AmtuPPX2L_L397Q_F420M_Gm::t-NOS and as selection marker a double mutated Arabidopsis AHAS-gene (AtAHASL_A122T_S653N), driven by its endogenous promotor and terminator, was used.

To combine Pti5, CaSAR (and PPO) expression cassettes in one vector a blunt ended fragment (restricted by PmeI and AsiSI) of PcUbi::Pti5_V84I::StCATHD-pA expression cassette was ligated into a SwaI and AsiSI cut vector that already contained the PcUbi::AmtuPPX2L_L397Q_F420M_Gm::Nos cassette, in a way that the PPO expression cassette is located downstream of the Pti5 expression cassette.

To add the CaSAR expression cassette to the PPO+Pti5 double stack, PPO+Pti5 double stack was excised from the above described vector as an SwaI/AsiSI fragment and ligated into an PmeI and AsiSI digested PcUbi::CaSAR::StCATHD-containing intermediate plasmid, in a way that the CaSAR expression cassette is located upstream of the Pti5+PPO fragement in a head to tail orientation (see FIG. 5).

As backbone a BiBAC (Binary Bacterial Artificial Chromosome) was used, which is based on the published vector pCH20 (C M Hamilton (1997) A binary-BAC system for plant transformation with high-molecular-weight DNA, Gene 1997 Oct. 24; 200(1-2):107-16.) The original pCH20 was modified by removal of the positive selection gene sacB from the T-DNA, and the addition of a p-AtAHASL::c-AtAHASL_A122T_S653N::t-AtAHAS selection expression cassette and the introduction of a new Multiple Cloning Sequence (MCS) to increase options for further cloning. The new MCS introduced the following eight-base cutters: SwaI, PacI, AscI, SbfI, FseI, AbsI, MreI, NotI, and PmeI.

The BiBAC backbone (as described above) was linearized with PmeI (5′GTTT/AAAC3′) and dephosphorylated to prevent self-ligation. The CaSAR+Pti5+PPO triple stacked fragment (see above) was excised with SwaI and PmeI. The resulting 6398 bp blunt fragment was then ligated to the linearized BiBAC backbone in way that the triple stacked fragment is located in the transfer-DNA region (between left and right border) downstream of the a p-AtAHASL::c-AtAHASL_A122T_S653N::t-AtAHAS selection expression cassette in a head to tail orientation. Therefore the final plant expression vector contains, in the T-DNA, at all 4 expression cassettes: p-AtAHASL::c-AtAHASL_A122T_S653N::t-AtAHAS (selection marker), p-PcUbi4-2::CaSAR_Gm::t-StCAT; p-PcUbi4-2::Pti5_V84I::t-StCAT and p-PcUbi4-2::AmtuPPX2L_L397Q_F420M_Gm::t-NOS.

Based on the result of the evaluation of resistance against soybean rust in T0 and/or T1 generation (see patent applications Pti5 und CASAR), the most resistance and phenotypically best looking 3-5 events were selected for further analysis.

Homozygous T2 to T6 seeds were used for field trials. To obtain homozygous seeds, segregating T1 seeds of the selected 3-5 events per construct were planted. Individual plants that were homozygous for the transgene were selected by using TaqMan® PCR assay as described by the manufacturer of the assay (Thermo Fisher Scientific, Waltham, MA USA 02451).

To prepare seeds for field trials 10-30 homozygous plants per event were grown in greenhouse under standard conditions (12 h daylength, 25° C.) and selfed (in-bred). Mature homozygous seeds were harvested approx. 120 days after planting. Harvested seeds of all 10-30 homozygous plants per event were pooled.

### Example 3 Generation of Transgenic Events

The expression vector construct (see example 1) was transformed into Agrobacterium rhizogenes using standard methods (e.g. see H. Klee. R. Horsch and S. Rogers 1987 Agrobacterium-Mediated Plant Transformation and its further Applications to Plant Biology; Annual Review of Plant Physiology Vol. 38: 467-486) A single colony was picked and checked for presence of the appropriate insert. An aliquot of the validated sample was handed over to soybean transformation.

### 3.1 Sterilization and Germination of Soy Seeds

Virtually any seed of any soy variety can be employed in the method of the invention. In this invention the soybean cultivar CD215 (Coodetec, Brazil) was used.

Soybean seeds from the cultivar CD215 were obtained from QC passed seed lots (CofA available in Section VII.a.). Discolored, cracked, and oblong seeds were removed to reduce bacterial and fungal contamination. Clean, round soybean seeds were placed in a single layer into a Petri dish and sterilized in a glass desiccator with a chlorine gas produced by adding 3.5 ml 12N HCl drop wise into 100 ml bleach. After 24 hours in the chamber, the top was removed, the gas allowed to escape, and the process repeated once more. After the second round of sterilization, the seeds were aired out in a sterile laminar flow hood for at least 30 minutes then closed and stored at room temperature in a locked cabinet until use. Approximately 65-70 seeds were plated on standard solid GM medium with or without 5 μM 6-benzyl-aminopurine (BAP) in 100 mm Petri dishes. Seedlings without BAP are more elongated and roots develop especially secondary and lateral root formation. BAP strengthens the seedling by forming a shorter and stockier seedling.

The seedlings were grown in the light (150-300 μm-2s-2) at +25° C. for approximately 8 days or until the epicotyl was extended beyond the cotyledons. The seedlings were used immediately for transformation once the desired stage was reached, or the seedlings were stored at 40 C overnight.

### 3.2—Growth and Preparation of Agrobacterium Culture

Agrobacterium cultures were prepared by streaking Agrobacterium rhizogenes carrying the above described vector (see example 2) onto solid YEP growth medium and incubating at 25. degree C. until colonies appeared (about 2 days). YEP media:10 g yeast extract. 10 g Bacto Peptone. 5 g NaCl. Adjust pH to 7.0, and bring final volume to 1 liter with H₂O, for YEP agar plates add 20 g Agar, autoclave).

For details and variants of the protocols see H. Klee. R. Horsch and S. Rogers 1987 Agrobacterium-Mediated Plant Transformation and its further Applications to Plant Biology; Annual Review of Plant Physiology Vol. 38:467-486). Depending on the selectable marker genes present on the Ti or Ri plasmid, the binary vector, and the bacterial chromosomes, different selection compounds were be used for A. tumefaciens and A. rhizogenes selection in the YEP solid and liquid media. Various Agrobacterium strains can be used for the transformation method.

After approximately two days, a single colony (with a sterile toothpick) was picked and 50 ml of liquid YEP was inoculated with antibiotics and shaken at 175 rpm (25° C.) until an OD600 between 0.8-1.0 is reached (approximately 2 d). Working glycerol stocks (15%) for transformation are prepared and one ml of Agrobacterium stock aliquoted into 1.5 ml Eppendorf tubes then stored at −80° C.

The day before explant inoculation, 200 ml of YEP were inoculated with 5 μl to 3 ml of working Agrobacterium stock in a 500 ml Erlenmeyer flask. The flask was shaken overnight at 25° C. until the OD600 was between 0.8 and 1.0. Before preparing the soy explants, the Agrobacteria were pelleted by centrifugation for 10 min at 5,500×g at 20° C. The pellet was resuspended in liquid CCM to the desired density (OD600 0.5-0.8) and placed at room temperature at least 30 min before use.

### 3.3—Explant Preparation and Co-Cultivation (Inoculation)

### 3.3.1 Method A: Explant Preparation on the Day of Transformation.

Seedlings at this time had elongated epicotyls from at least 0.5 cm but generally between 0.5 and 2 cm. Elongated epicotyls up to 4 cm in length had been successfully employed. Explants were then prepared with: i) with or without some roots, ii) with a partial, one or both cotyledons, all preformed leaves were removed including apical meristem, and the node located at the first set of leaves was injured with several cuts using a sharp scalpel.

This cutting at the node not only induced Agrobacterium infection but also distributed the axillary meristem cells and damaged pre-formed shoots. After wounding and preparation, the explants were set aside in a Petri dish and subsequently co-cultivated with the liquid CCM/Agrobacterium mixture for 30 minutes. The explants were then removed from the liquid medium and plated on top of a sterile filter paper on 15×100 mm Petri plates with solid co-cultivation medium. The wounded target tissues were placed such that they are in direct contact with the medium.

### 3.3.2 Modified Method A: Epicotyl Explant Preparation

Soyepicotyl segments prepared from 4 to 8 d old seedlings were used as explants for regeneration and transformation. Seeds of soya cv. L00106CN, 93-41131 and Jack were germinated in 1/10 MS salts or a similar composition medium with or without cytokinins for 4 to 8 d. Epicotyl explants were prepared by removing the cotyledonary node and stem node from the stem section. The epicotyl was cut into 2 to 5 segments. Especially preferred are segments attached to the primary or higher node comprising axillary meristematic tissue.

The explants were used for Agrobacterium infection. Agrobacterium AGL1 harboring a plasmid with the gene of interest (GOI) and the AHAS, bar or dsdA selectable marker gene was cultured in LB medium with appropriate antibiotics overnight, harvested and resuspended in a inoculation medium with acetosyringone. Freshly prepared epicotyl segments were soaked in the Agrobacterium suspension for 30 to 60 min and then the explants were blotted dry on sterile filter papers. The inoculated explants were then cultured on a co-culture medium with L-cysteine and TTD and other chemicals such as acetosyringone for increasing T-DNA delivery for 2 to 4 d. The infected epicotyl explants were then placed on a shoot induction medium with selection agents such as imazapyr (for AHAS gene), glufosinate (for bar gene), or D-serine (for dsdA gene). The regenerated shoots were subcultured on elongation medium with the selective agent.

For regeneration of transgenic plants the segments were then cultured on a medium with cytokinins such as BAP, TDZ and/or Kinetin for shoot induction. After 4 to 8 weeks, the cultured tissues were transferred to a medium with lower concentration of cytokinin for shoot elongation. Elongated shoots were transferred to a medium with auxin for rooting and plant development. Multiple shoots were regenerated.

Many stable transformed sectors showing strong cDNA expression were recovered. Soybean plants were regenerated from epicotyl explants. Efficient T-DNA delivery and stable transformed sectors were demonstrated.

### 3.3.3 Method B: Leaf Explants

For the preparation of the leaf explant the cotyledon was removed from the hypocotyl. The cotyledons were separated from one another and the epicotyl is removed. The primary leaves, which consist of the lamina, the petiole, and the stipules, were removed from the epicotyl by carefully cutting at the base of the stipules such that the axillary meristems were included on the explant. To wound the explant as well as to stimulate de novo shoot formation, any pre-formed shoots were removed and the area between the stipules was cut with a sharp scalpel 3 to 5 times.

The explants are either completely immersed or the wounded petiole end dipped into the Agrobacterium suspension immediately after explant preparation. After inoculation, the explants are blotted onto sterile filter paper to remove excess Agrobacterium culture and place explants with the wounded side in contact with a round 7 cm Whatman paper overlaying the solid CCM medium (see above). This filter paper prevents A. tumefaciens overgrowth on the soy-explants. Wrap five plates with Parafilm™. “M” (American National Can, Chicago, Ill., USA) and incubate for three to five days in the dark or light at 25° C.

### 3.3.4 Method C: Propagated Axillary Meristem

For the preparation of the propagated axillary meristem explant propagated 3-4 week-old plantlets were used. Axillary meristem explants can be pre-pared from the first to the fourth node. An average of three to four explants could be obtained from each seedling. The explants were prepared from plantlets by cutting 0.5 to 1.0 cm below the axillary node on the internode and removing the petiole and leaf from the explant. The tip where the axillary meristems lie was cut with a scalpel to induce de novo shoot growth and allow access of target cells to the Agrobacterium. Therefore, a 0.5 cm explant included the stem and a bud.

Once cut, the explants were immediately placed in the Agrobacterium suspension for 20 to 30 minutes. After inoculation, the explants were blotted onto sterile filter paper to remove excess Agrobacterium culture then placed almost completely immersed in solid CCM or on top of a round 7 cm filter paper overlaying the solid CCM, depending on the Agrobacterium strain. This filter paper prevents Agrobacterium overgrowth on the soy-explants. Plates were wrapped with Parafilm™. “M” (American National Can, Chicago, Ill., USA) and incubated for two to three days in the dark at 25° C.

### 3.4—Shoot Induction

After 3 to 5 days co-cultivation in the dark at 25° C., the explants were rinsed in liquid SIM medium (to remove excess Agrobacterium) (SIM, see Olhoft et al 2007 A novel Agrobacterium rhizogenes-mediated transformation method of soy using primary-node explants from seedlings In Vitro Cell. Dev. Biol.—Plant (2007) 43:536-549; to remove excess Agrobacterium) or Modwash medium (1×B5 major salts, 1×B5 minor salts, 1×MSIII iron, 3% Sucrose, 1×B5 vitamins, 30 mM MES, 350 mg/L Timentin pH 5.6, WO 2005/121345) and blotted dry on sterile filter paper (to prevent damage especially on the lamina) before placing on the solid SIM medium. The approximately 5 explants (Method A) or 10 to 20 (Methods B and C) explants were placed such that the target tissue was in direct contact with the medium. During the first 2 weeks, the explants could be cultured with or without selective medium. Preferably, explants were transferred onto SIM without selection for one week.

For leaf explants (Method B), the explant should be placed into the medium such that it is perpendicular to the surface of the medium with the petiole imbedded into the medium and the lamina out of the medium.

For propagated axillary meristem (Method C), the explant was placed into the medium such that it was parallel to the surface of the medium (basipetal) with the explant partially embedded into the medium.

Wrap plates with Scotch 394 venting tape (3M, St. Paul, Minn., USA) were placed in a growth chamber for two weeks with a temperature averaging 25° C. under 18 h light/6 h dark cycle at 70-100 μE/m2s. The explants remained on the SIM medium with or without selection until de novo shoot growth occurred at the target area (e.g., axillary meristems at the first node above the epicotyl). Transfers to fresh medium can occur during this time. Explants were transferred from the SIM with or without selection to SIM with selection after about one week. At this time there was considerable de novo shoot development at the base of the petiole of the leaf explants in a variety of SIM (Method B), at the primary node for seedling explants (Method A), and at the axillary nodes of propagated explants (Method C).

Preferably, all shoots formed before transformation were removed up to 2 weeks after co-cultivation to stimulate new growth from the meristems. This helped to reduce chimerism in the primary transformant and increase amplification of transgenic meristematic cells. During this time the explant may or may not be cut into smaller pieces (i.e. detaching the node from the explant by cutting the epicotyl).

### 3.5—Shoot Elongation

After 2 to 4 weeks (or until a mass of shoots was formed) on SIM medium (preferably with selection), the explants were transferred to SEM medium (shoot elongation medium, see Olhoft et al., a novel Agrobacterium rhizogenes-mediated transformation method of soy using primary-node explants from seedlings. In Vitro Cell. Dev. Biol.—Plant (2007) 43:536-549) that stimulates shoot elongation of the shoot primordia. This medium may or may not contain a selection compound.

After every 2 to 3 weeks, the explants were transfer to fresh SEM medium (preferably containing selection) after carefully removing dead tissue. The explants should hold together and not fragment into pieces and retain somewhat healthy. The explants were continued to be transferred until the explant dies or shoots elongate. Elongated shoots>3 cm were removed and placed into RM medium for about 1 week (Method A and B), or about 2 to 4 weeks depending on the cultivar (Method C) at which time roots began to form. In the case of explants with roots, they were transferred directly into soil. Rooted shoots were transferred to soil and hardened in a growth chamber for 2 to 3 weeks before transferring to the greenhouse. Regenerated plants obtained using this method were fertile and produced on average 500 seeds per plant.

After 5 days of co-cultivation with Agrobacterium tumefaciens transient expression of the gene of interest (GOI) was widespread on the seedling axillary meristem explants especially in the regions wounding during explant preparation (Method A). Explants were placed into shoot induction medium without selection to see how the primary-node responds to shoot induction and regeneration. Thus far, greater than 70% of the explants were formed new shoots at this region. Expression of the GOI was stable after 14 days on SIM, implying integration of the T-DNA into the soybean genome. In addition, preliminary experiments resulted in the formation of cDNA expressing shoots forming after 3 weeks on SIM.

For Method C, the average regeneration time of a soybean plantlet using the propagated axillary meristem protocol was 14 weeks from explant inoculation. Therefore, this method has a quick regeneration time that leads to fertile, healthy soybean plants.

### Example 4: Multiplication of Seeds in Greenhouse

Homozygous T2 to T6 seeds were used in this application. To obtain homozygous seeds, segregating T1 seeds of the selected events (as described in Example 3) were planted. Individual plants that were homozygous for all 4 transgenes (AHAS, CaSAR, Pti5, PPO were selected by using TaqMan® PCR assay as described by the manufacturer of the assay (Thermo Fisher Scientific, Waltham, MA USA 02451).

2—100 Homozygous plants per event and generation were grown under standard conditions (12 h daylength, 25° C.) and selfed (in-bred). Mature homozygous seeds were harvested approx. 120 days after planting. The same methods and conditions were used for each following generation.

In each generation, one plant was selected based on phenotypic and molecular characteristics to deliver the founder seeds for the next generation (single seed decent). Harvested seeds of all other homozygous plants per event were pooled for further analysis.

### Example 5: Field Trials

Homozygous T2 to T6 seeds of 4 events of the construct expressing Pti5 and SAR8.2 were tested in the field for resistance against various soybean diseases. As the incidence and severity of soybean diseases (other than Asian soybean rust) is strongly depended on environmental and weather conditions, a broad testing on many sites across Brazil and US was performed. The occurrence and severity of all soybean diseases was recorded. Diseases occurred with a relevant severity at the sites in Uberlandia (MG), Ponta Grossa (PR), Londrina (PR), Sinop (MT), Trindade (RJ) and Primavera do L'Este (MT).

Material was tested in split plot trials (2 m long, 4 rows per plot), 3-4 replications per event and trial site. About 10% of the plots were used as control. The untransformed wild-type (WT) mother line was used as control. Field trials to test trait performance were grown using standard cultural practice, e.g. in terms of weed and insect control and fertilization but no fungicide was applied to allow infestation of the field trials by multiple fungal diseases. Rating of the diseased leaf area per disease was performed at various dates across the season by the eye of experienced personnel. Exact dates of the individual ratings depend on the progress of the respective disease infection, which is strongly depended on environmental factors that are strongly different at the different field trial sites.

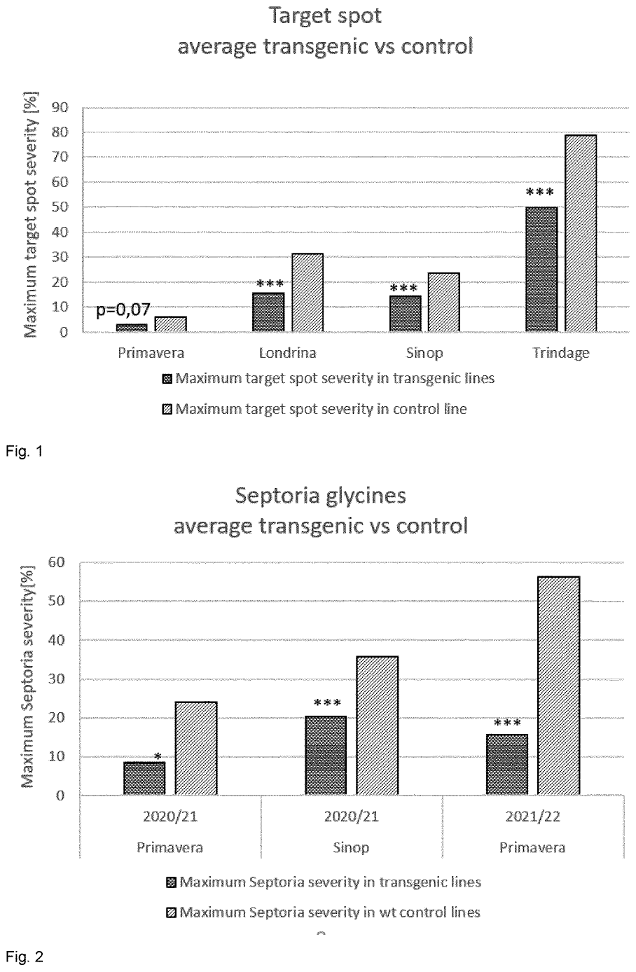

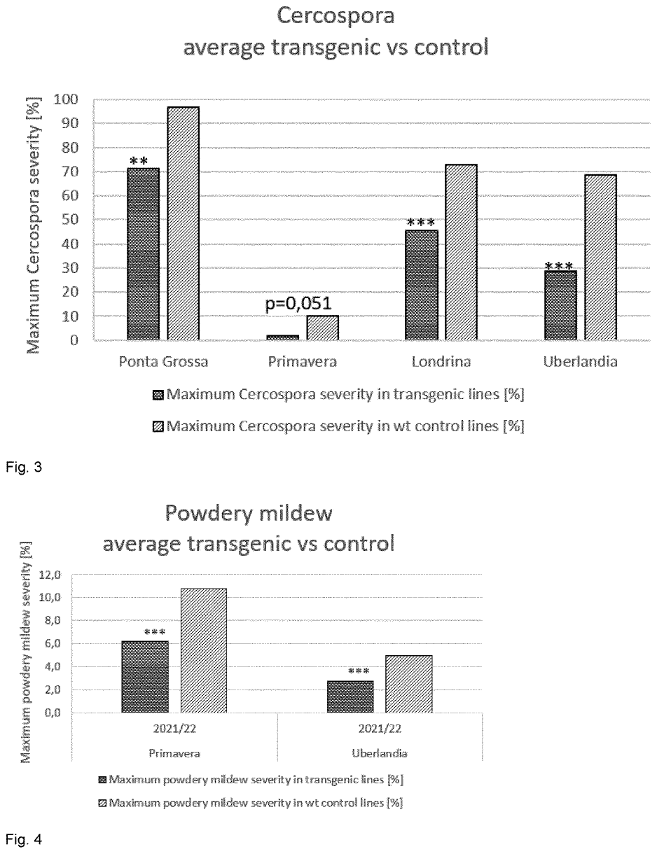

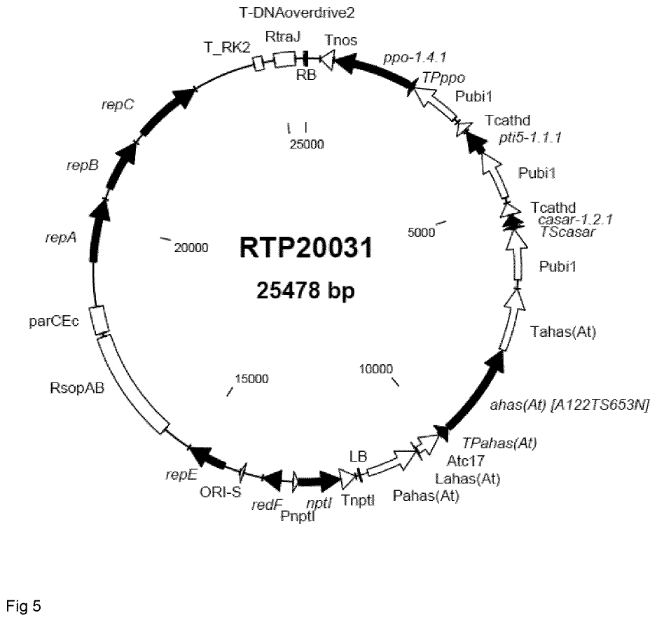

It could be shown that the expression of Pti5 and SAR8.2 leads to a broad spectrum disease resistance against target spot, Septoria brown spot, Cercospora leaf blight and powdery mildew, (see FIGS. 1-4).

### Example 6: Observations

Field trials were also performed in several locations in across US using the same event expressing Pti5 and SAR8.2. To adapt the event to the local environmental conditions in US, the event was backcrossed to locally adapted varieties (e.g. with lower maturity groups.) Field trials were performed in multiple locations with a focus on those regions where soybeans are grown, but also in some more southern states. In some of those field trials we observed a strong disease pressure of Sudden death syndrome (SDS), Frogeye leaf spot and Phyllosticta leaf spot, if not treated with fungicides. In those field trials we also observed a considerably lower disease severity of SDS, Frogeye leaf spot and Phyllosticta leaf spot in the events expressing of Pti5 and SAR8.2.

In those trials It could be shown that the expression of Pti5 and SAR8.2 leads to a broad spectrum disease resistance against all of SDS, Frogeye leaf spot and Phyllosticta leaf spot.

1. Method for conferring, modifying or increasing resistance against one or multiple pathogen infections of a plant, plant part, or plant cell in comparison to a respective wild type plant, wild type plant part, or wild type plant cell, wherein the pathogens are selected from
the following taxa: (1) order Pleosporales, (2) order Mycosphaerellales, (3) order Erysiphales, (4) order Botryosphaeriales and/or
the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot,
wherein the method comprises the step of increasing the production and/or accumulation of a combination of at least a Pti5 protein and a CaSAR protein, and/or a Pti5-CaSAR fusion protein, in the plant, plant part or plant cell.

2. Method according to claim 1, wherein the Pti5 protein
codes for a protein comprising an apetala 2 domain as explained in PFAM entry PF00847 and binding to the Pti5 GCC box, and/or
has at least 72% sequence identity to SEQ ID NO. 1.

3. Method according to claim 1, wherein the CaSAR protein
comprises or consists of a SAR8.2 domain according to PFAM entry PF03058, and/or
has at least 60% sequence identity to SEQ ID NO. 2.

4. Method according to claim 1, wherein the Pti5-CaSAR fusion protein comprises a Pti5 protein domain according to claim 2 linked, via a linker, to a CaSAR protein domain according to claim 3.

5. Method according to claim 1, comprising the steps of
i) stably transforming a plant cell with
a) a heterologous expression cassette comprising a Pti5 gene and, optionally in a separate transformation step, with a heterologous expression cassette comprising a CaSAR gene, and/or
b) a heterologous expression cassette comprising a Pti5-CaSAR fusion gene,
ii) regenerating a plant from the plant cell, and
iii) expressing the Pti5, CaSAR and/or Pti5-CaSAR fusion proteins in cells of said plant.

6. Method according to claim 1, wherein the plant comprises a heterologous Pti5 expression cassette and a heterologous CaSAR expression cassette, wherein for each expression cassette the respective Pti5 or CaSAR gene is operably linked to any of
a) a constitutively active promoter,
b) a tissue-specific or tissue-preferred promoter,
c) a promoter inducible by exposition of the plant to a pest.

7. Method of producing a crop plant showing improved resistance against one or multiple pathogens compared to the corresponding wild type, comprising the steps of
i) crossing two different crop plants, wherein at least one of the plants comprises a Pti5 gene and a CaSAR gene, and/or a Pti5-CaSAR fusion gene, to produce members of a filial generation,
ii) detecting the presence of a polynucleotide indicative of the Pti5, CaSAR and/or Pti5-CaSAR fusion protein and in said members of the filial generation, and
iii) selecting at least one member of the filial generation with confirmed presence of the polynucleotide indicative of the Pti5, CaSAR and/or Pti5-CaSAR fusion protein,
wherein the pathogens are selected from
the following taxa: (1) order Pleosporales, (2) order Mycosphaerellales, (3) order Erysiphales, (4) order Botryosphaeriales, and/or
the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot.

8. Method of reducing or preventing single or multiple pathogen pressure of a field, comprising the step of cultivating, on said field, plants comprising
a) a heterologous expression cassette comprising a Pti5 gene and a heterologous expression cassette comprising a CaSAR gene, and/or
b) a heterologous expression cassette comprising a Pti5-CaSAR fusion gene,
wherein the pathogens are selected from
the following taxa: (1) order Pleosporales, (2) order Mycosphaerellales, (3) order Erysiphales, (4) order Botryosphaeriales, and/or
the causative agents of the following diseases: (1) target spot, (2) Cercospora leaf blight, (3) Septoria infection and (4) powdery mildew, (5) Frogyeye leaf spot, (6) Sudden Death Syndrome and (7) Phyllosticta leaf spot.

9. Method according to claim 1, wherein the plant is a crop plant.

10. (canceled)
